(19) United States
(12) Patent Application Publication
(10) Pub. No.: US 2024/0050618 A1
(43) Pub. Date: Feb. 15, 2024

(54) ULTRAVIOLET AIRFLOW TREATMENT SYSTEM

(71) Applicant: Peter KOCHAN, Bosaca (SK)

(72) Inventor: Peter KOCHAN, Bosaca (SK)

(21) Appl. No.: 18/268,252

(22) PCT Filed: Dec. 18, 2020
(86) PCT No.: PCT/SK2020/050021

§ 371 Date: Jun. 19, 2023

(51) Int. Cl. A61L 9/20; B08B 7/00; A61M 16/10; A61M 16/06

(52) U.S. Cl. CPC A61L 9/20; B08B 7/0057; A61M 16/10; A61M 16/0666; A61L 2209/12; A61L 2209/11; A61M 2205/04; A61M 2205/3592; A61M 2205/50; A61M 2205/8243

(57) ABSTRACT

Miniaturized high-intensity shortwave UV airflow treatment system in a compact easy-to-wear and fully portable form factor. Practical, germicidal air treatment system may be worn by or be carried by a user to kill or deactivate germs, viruses or other pathogens, which are located in the air to be breathed by the user. Air being inhaled or exhaled by the user is exposed to Ultra-Violet C-band (UVC) radiation. This UVC radiation is lethal to undesirable germs, viruses and other pathogens. In this manner, pathogen-free air is being inhaled or exhaled by the user.

### TECHNICAL FIELD

The invention generally relates to using ultraviolet radiation to disinfect air.

### BACKGROUND ART

Ultraviolet (UV) light is electromagnetic radiation with wavelengths shorter than visible tight but longer than X-rays. UV is categorized into several wavelength ranges, with short-wavelength UV (UVC) being between 100-280 nm, out of which 250-280 nm is typically considered “germicidal UV”, because it kills pathogens, primarily by damaging their DNA. Ultraviolet germicidal irradiation for the purpose of disinfection has been an accepted practice since mid-20th century in medical field, or for drinking and wastewater sterilization. High-intensity shortwave ultraviolet light is commonly used for disinfecting smooth surfaces such as dental tools. Unfortunately, existing UV-light sources for biocidal applications are typically fluorescent UV light bulbs, deuterium or xenon-arc excimer lamps. These lamps and bulbs are large and usually require the presence of a ballast and AC power source for stable operation. See, for example, U.S. Pat. No. 5,817,276 assigned to Steril-Aire. Size and power requirements of such high-intensity shortwave ultraviolet disinfectant systems make them unsuitable for portable use and are not known in this context.

Even with Introduction of UV light-emitting diodes (LEDs) and other advancements in technology miniaturization, conventional germicidal UV light applications use these UV light sources in 250 nm-280 nm wavelength, allowing them to disinfect unoccupied spaces or enclosed areas, however using them in close proximity to humans is not possible since it is well known that exposure to these wavelengths is a health hazard, causing eye diseases, skin cancer and other health problems.

Attempted portable solutions in this field include a protective face mask combining mechanical filtering element with 265 nm UV-C LEDs, however due to the health hazard associated with the 265 nm UV wavelength, the UV light source must be physically enclosed in a chamber, restricting the airflow, and only disinfecting the air passing through this chamber, whereas if the mask is not property face-fitted, a significant amount of air can leak through the periphery of the mask allowing Inhalation of airborne pathogens by the person wearing it, significantly reducing the mask's effectiveness and increasing the risk of respiratory infection. Many facemasks and respirators cannot be re-used repeatedly, and their disposal Is problematic and poses potential environmental danger and burden. Furthermore, facemasks and respirators are generally uncomfortable to wear, which may discourage people from using them, they usually muffle the voice and can make speech less intelligible, the mechanical or other filtering elements in them usually only capture some of the pathogens and need frequent cleaning or replacement, and some people are unable to wear facemasks and respirators due to breathing difficulties, allergies, or other reasons.

From the discussion above it should be apparent that there is a need for air treatment against airborne viral or bacterial infection that provides protection for persons in a variety of environments as they move about, without having to wear a facemask or other protective device that could pose an obstruction to breathing or speech intelligibility. The present Invention satisfies this need.

### SUMMARY OF INVENTION

Portable air treatment system that is worn or carried by a user and irradiates the airflow with energy in the electromagnetic spectrum, while the air Is being inhaled by the user. The irradiated energy is sufficient to substantially cleanse the Inhaled air of most airborne viruses and bacteria.

Recent third-party research has shown that the far-UVC range of 200 nm-230 nm has similar anti-microbial and anti-viral properties as conventional germicidal UV lights, but without producing the corresponding negative health effects. Reason for this is that light in this far-UVC wavelength range has a very limited penetration depth, and is very strongly absorbed by proteins through the peptide bond and other biomolecules. Therefore its ability to penetrate biological materials is very limited compared with, for example, 254 nm (or higher) conventional germicidal UV light. Although the penetration is limited, it is still much larger than the size of viruses and bacteria, therefore far-UVC tight is as efficient in killing these pathogens as conventional germicidal UV light. However, unlike 250-300 nm germicidal UV light, far-UVC 200-230 nm light cannot penetrate either the human stratum corneum (the outer dead-cell skin layer), nor the ocular tear layer, nor even the cytoplasm of individual human cells. Thus, far-UVC light does not pose a health hazard since it cannot reach or damage living cells in the human skin or the human eye, in contrast to the conventional germicidal UV light which can reach these sensitive cells. (Buonanno, M., Welch, D., Shuryak, I. et al. Far-UVC light (222 nm) efficiently and safely inactivates airborne human coronaviruses. Sci Rep 10, 10285 (2020). https://doi.org/10.1038/s41598-020-67211-2)

Recent Independent studies at Columbia University and Kobe University have also confirmed that shortwave far-UVC photons at 200-230 nm range have germicidal effect without penetrating or damaging live human cells, making far-UVC sanitizing light safe for humans, and since potentially harmful ozone is only produced at wavelengths below 200 nm, the range of 200-230 nm can be safely used in air treatment and personal protection devices.

Presented invention applies this knowledge in an air treatment system that uses light in the 200-230 nm far-UVC range to irradiate and disinfect the air being inhaled or exhaled by the user, and presented embodiments represent some of the potential industrial applications in the form of germicidal disinfection air treatment devices that reduce risk from airborne pathogens.

Accordingly, it Is an object of the present invention to provide a UVC air treatment system for killing germs, viruses, and other pathogens through the use of radiation in a compact and portable form factor.

It Is still another object of the present invention to minimize the obstructions to airflow of inhaled or exhaled air of the user of the germicidal airflow treatment system.

It Is still another object of the present invention to minimize exposure to airborne pathogens for anyone in close proximity to an already infected user by disinfecting the air being exhaled by the user of the germicidal airflow treatment system.

It is still another object of the present invention to allow total freedom of movement of the user of the germicidal airflow treatment system.

These and other features and advantages of the present invention further enhancing and advancing the technology in the art will become apparent upon further review of the following specification, drawings, and claims. It should be understood, however, that the detailed description and the specific examples while indicating preferred embodiments of the invention are given by way of illustration only, since various changes and modifications within the spirit and scope of the invention will become apparent to those skilled in the art from this detailed description.

Embodiments of the invention pertain to a portable system that is worn or carried by a user and irradiates the airflow with energy in the UVC electromagnetic spectrum while the air is being inhaled by the user. The irradiated energy is sufficient to substantially cleanse the air of airborne viruses and the like prior to Inhalation. The system includes a portable radiation source with a power supply to generate the energy for irradiation. The airflow is irradiated while entering and passing through the airways of the upper respiratory system (nose, mouth, throat, pharynx) such that airborne germs in the airflow are substantially destroyed or rendered harmless before the airflow enters lower respiratory system and germs in it potentially cause an infection. Viral and bacterial exposure is defeated at the point of inhalation, thus reducing the threat of infection independent of the airborne bacteria type, virus strain or mutation of the virus.

In one aspect of the system, electromagnetic radiation for the air treatment is generated at the UVC light source and is directed from the light source to upper airways by a conduit such as fiber optics. The Irradiation can occur through light-diffusing fiber used for directing UVC light from the UVC source, or through an exposed end of the fiber optics, or through means such as a fiberglass mat, or a fiber optic weave, or a fiber optic rod, or a glass disk, or a diffuser, or a terminus in the upper airways, coupled to the fiber optics and configured so that the airflow in the upper respiratory tract must pass it by.

Source for the UVC light for air treatment in various embodiments can optionally be a UVC laser, a UVC lamp, a UVC light emitting diode, a UVC light emitting semiconductor, a UVC microplasma lamp or array, or a frequency-doubled laser generating light in the UVC spectrum, in a portable form factor that can be carried by the user.

Apparatus can be powered by a standard or a rechargeable battery, optionally by any other suitable power source so as to preferably provide a power capacity of at least 1 microwatt-hour. If a rechargeable battery is used, it can be recharged through a charging port or wirelessly using electromagnetic induction. Alternatively, the apparatus can be powered by an external power supply by wireless power transfer.

Function of the system is managed by an electronics module, which may comprise a computer, optionally also an antenna to communicate with external devices, allowing the user to control and manage the airflow treatment system both directly and indirectly by an externally connected device such as smartphone. This also allows a function of automatically turning on the airflow treatment system when leaving biosafe environment such as personal home or car, or when entering potentially hazardous environment. System functionality or power source status can be confirmed by either integrated visual or acoustic indicators, or remotely by means of externally connected device such as a smartphone.

In yet another embodiment of the system some or all of the components may be implanted in the body of the user. For example, the main apparatus body may be located subcutaneously near the chest muscle with the flexible conduit passing through the soft neck tissues into the trachea and pharynx, with the battery or power source in the apparatus adapted for wireless charging. Alternatively, only the flexible conduit may be implanted with the distal end in the upper airways of the user, and the coupling element at the proximal end protruding out of the skin for example near the collarbone, allowing coupling with the main apparatus worn or carried by the user.

In some embodiments, one or more of the apparatuses, systems or devices described herein can be used, and their use in combination with other forms of disinfection such as 250-450 nm photo-disinfection or mechanical filters are within the scope of embodiments of the invention.

### Technical Problem

Personal protection against airborne viral or bacterial Infection currently requires wearing a facemask or a respirator, and existing ultraviolet disinfection devices are usually large, cannot be worn on body of the user, and use ultraviolet light wavelengths that are harmful to humans.

### Solution to Problem

Miniaturized battery-powered high-intensity 200-230 nm shortwave UV airflow treatment system in a compact easy-to-wear and fully portable form factor that may be worn by or be carried by a user to kill or deactivate germs, viruses or other pathogens, which are located in the air to be breathed by the user.

### Advantageous Effects of Invention

Invention provides protection for persons in a variety of environments as they move about.

Invention provides personal protection against airborne pathogens without having to wear a facemask posing an obstruction to breathing.

Invention provides personal protection against airborne pathogens without having to wear a facemask that makes speech less intelligible.

Invention provides protection for persons in a vicinity of an Infected person exhaling air containing pathogens.

### BRIEF DESCRIPTION OF DRAWINGS

Various other objects, features and attendant advantages of the present Invention will become fully appreciated as the same becomes better understood when considered in conjunction with the accompanying drawings, in which like reference characters designate the same or similar parts throughout the several views.

For a better understanding of the embodiments described herein and to show more clearly how they may be carried into effect, reference will now be made, by way of example only, to the accompanying drawings which show at least one exemplary embodiment, and in which:

FIG. 1 is a perspective illustration of an embodiment where the apparatus body is attached to glasses worn by the user.

FIG. 2 is a perspective illustration of an embodiment where the apparatus body is attached to a neck strap worn by the user.

FIG. 3 is a cross sectional view of the body of the user, in an embodiment where the air treatment system is fully implanted inside the user's body.

### DESCRIPTION OF EMBODIMENTS

Unless otherwise defined, scientific and technical terms used in connection with the present disclosure shall have the meanings that are commonly understood by those of ordinary skill in the art. Further, unless otherwise required by context, singular terms shall include pluralities and plural terms shall Include the singular.

In the following description, specific details are set out to provide examples of the claimed subject matter. However, the embodiments described below are not intended to define or limit the claimed subject matter.

It will be appreciated that, for simplicity and clarity of illustration, where considered appropriate, reference numerals may be repeated among the figures to Indicate corresponding or analogous elements or steps. Numerous specific details are set forth in order to provide a thorough understanding of the exemplary embodiments of the subject matter described herein. However, it will be understood by those of ordinary skill in the art that the embodiments described herein may be practiced without these specific details. In other Instances, well-known methods, procedures and components have not been described in detail so as not to obscure the present subject matter. Furthermore, this description is not to be considered as limiting the scope of the subject matter in any way but rather as illustrating the various embodiments.

Indeed, it should be understood that various changes may be made in the form, details, arrangement and proportions of the parts. Such changes do not depart from the scope of the invention which comprises the matter shown and described herein and set forth in the appended claims.

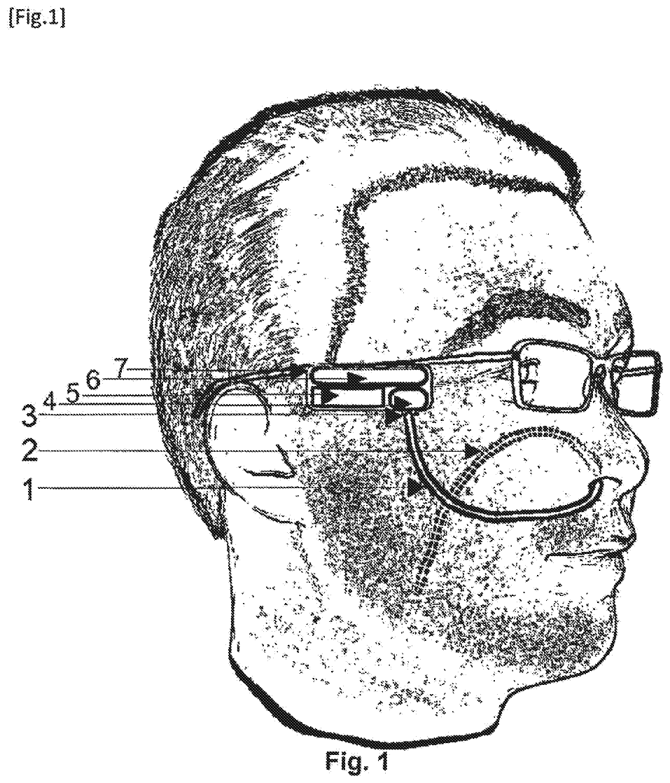

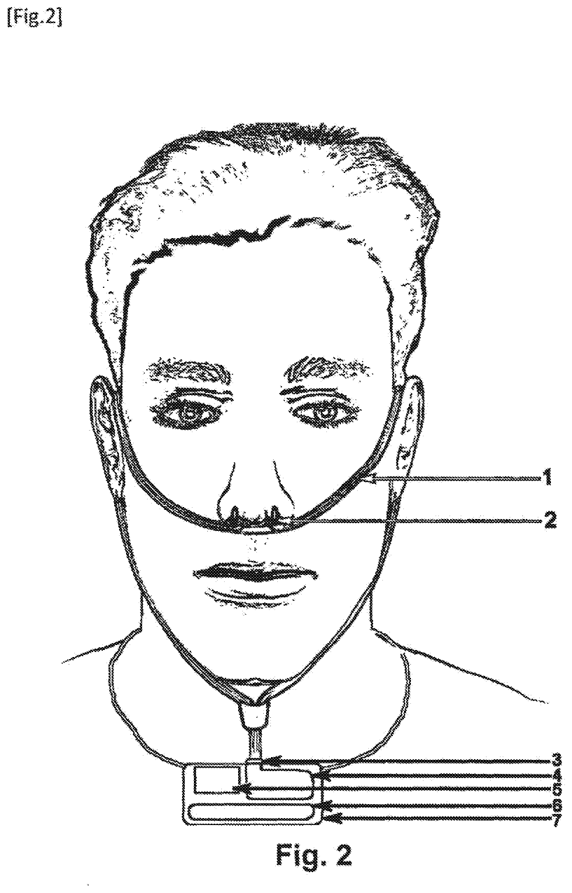

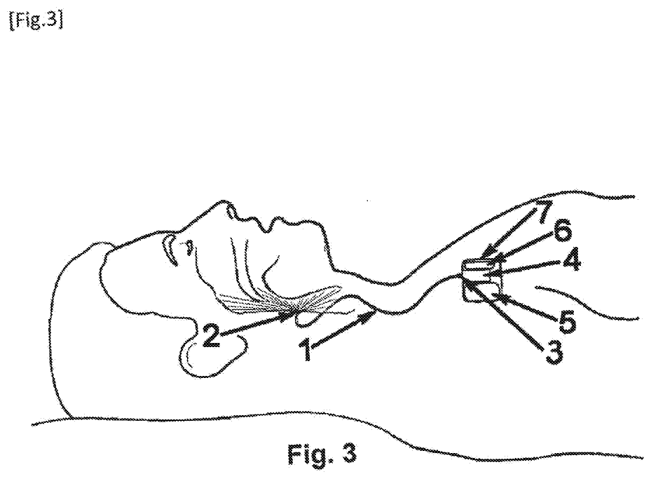

As shown on [FIG. 1], [FIG. 2], and [FIG. 3], the air treatment system comprises of the main body of the apparatus 7, a battery or other power supply 6, electronics module 5, irradiation source 4, coupling element 3, light diffusing element 2, and a flexible conduit 1.

In an embodiment shown on [FIG. 1], the main body of the apparatus 7 may be an oval shaped plastic casing about 70 mm in length, 20 mm in width, and 35 mm in depth, attached to the side frame of user's glasses, and containing a battery 6. for example an 18650 type lithium-ion battery, electronics module 5 with an external power switch, a charging port, and a LED light serving as visual function indicator, optionally comprising a computer and an antenna for communication with external device such as smartphone, irradiation source 4 may be a laser outputting light in the 222 nm UVC wavelength, coupling element 3 connects the main apparatus body with the proximal end of the flexible conduit 1, wherein this conduit is passing alongside user's cheek into the nose, directing the radiation toward the UVC light side-emitting optical fiber 2 at the distal end located in the user's nose and pharynx. Optionally, this side-emitting optical fiber can be a Corning® or Fibrance® light diffusing fiber.

In an embodiment shown on [FIG. 2], the main body of the apparatus 7 may be an oval shaped plastic casing about 70 mm in length, 20 mm in width, and 35 mm in depth, carried on a strap around user's neck, containing a battery 6, for example an 18650 type lithium-ion battery, electronics module 5 which may comprise a computer, an antenna, an external power switch, a charging port, and a LED light serving as visual function indicator, irradiation source 4 may be an array of multitude of UVC light emitting diodes outputting light in the 200-230 nm UVC spectrum optically focused into the coupling element 3 connecting the main apparatus body with the proximal ends of two flexible conduits 1, wherein these conduits are passing behind user's ears and then alongside user's cheeks into the nose, directing the radiation toward the tubular shaped fiber optic mat nose inserts 2 with opaque outer sides at the distal end located in user's nostrils. with a yielding bridge piece connecting the two conduits under the nose and holding the fiber optic mat inserts securely in the nostrils of the user.

In an embodiment shown on [FIG. 3], the main body of the apparatus 7 may be an oval shaped casing about 70 mm in length, 20 mm in width, and 10 mm in depth made from plastic biocompatible material, surgically implanted subcutaneously near the chest muscle, containing a battery 6, electronics module 5 comprising of a computer and an antenna, irradiation source 4 may be a frequency-doubled laser generating light in the UVC 222 nm wavelength, with the coupling element 3 connecting the main apparatus body with the proximal end of a flexible conduit 1 passing through the soft neck tissues into the trachea, directing the radiation toward the luminous diffuser 1 at the distal end located in user's pharynx. Optionally, this diffuser can be a SCHOTT® spherical diffuser.

It will of course be appreciated by those skilled in the art that many variations of the described embodiments would be possible within the scope of the invention defined by the claims herein.

### Examples

Commercially sourced 5 Watt 450 nm handheld laser, attached to user's glasses frame, powered by 18650 battery and coupled with frequency-doubling BBO crystal producing through second-harmonic generation UVC light in the 222 nm wavelength, optically coupled by a multimode fiber coupler with a biconvex lens reducing the output beam diameter into a 400 μm/440 μm/480 μm (core/cladding/coating) solarization resistant fiber, on the distal end with a diffuser made of fused silica inscribed with micro-grooves, produces sufficient far-UVC irradiance to achieve significant inactivation (D90 or more) of most airborne Coronaviridae viruses at the exposure rate required for the volume and speed of air passing through upper airways.

Dose required to kill viruses and bacteria in the air with far-UVC is slightly higher than with conventional UVC at 254 nm; however, the lack of harmful biological effects with far-UVC allows the possibility to safely use it in close proximity to human skin and inside human upper airways.

Recent advances in far-UVC laser technology have shown promise for further shrinking both the size and the cost of the laser modules to be better suited for widespread adoption, and latest developments in the light diffusing technology, such as the new glass diffusers made by SCHOTT, which are better suited for this application, as well as developments in minimizing solarization effects reducing UVC transmittance in optical fibers will further enhance system efficiency, allowing smaller system size and requiring lower power consumption as well as longer runtime per battery charge.

### INDUSTRIAL APPLICABILITY

Invention can be used in any kind of industry, by anyone needing protection from airborne pathogens, whether in medical industry, manufacturing, agriculture, forestry, livestock breeding, fisheries, mining, processing industries and services:

### REFERENCE SIGNS LIST

### Reference to Deposited Biological Material

### Sequence Listing Free Text

### CITATION LIST

Buonanno, M., Welch, D., Shuryak, I. et al. Far-UVC fight (222 nm) efficiently and safely inactivates airborne human coronaviruses. Sci Rep 10, 10285 (2020). https://doi.org/10.1038/s41598-020-67211-2

### PATENT LITERATURE

PTL1:

### Non Patent Literature

NPL1:

1. A personal wearable airflow treatment system, consisting of:
at least one portable apparatus containing at least one light source emitting electromagnetic radiation in far-UVC wavelength range of 200-230 nm, coupled to at least one optical fiber extending therefrom, wherein electromagnetic radiation is by means of the optical fiber directed toward air being inhaled or exhaled by a person, treating the air with a radiated energy of the electromagnetic spectrum reducing the amount of pathogens being inhaled or exhaled by the person,
an electronic control module,
a power supply,
a wearable housing.

2. (canceled)

3. (canceled)

4. The airflow treatment system as defined in claim 1, wherein the apparatus is powered by a non-rechargeable battery inside the housing.

5. The airflow treatment system as defined in claim 1, wherein the apparatus is powered by a rechargeable power source inside the housing.

6. The airflow treatment system as defined in claim 1, wherein the apparatus is powered by an external power supply.

7. (canceled)

8. (canceled)

9. (canceled)

10. The airflow treatment system as defined in claim 1, wherein the fiber optic includes an area of reduced transmissivity and an exposed surface from which the electromagnetic radiation is emitted.

11. The airflow treatment system as defined in claim 1, wherein the system comprises a conduit that directs the electromagnetic energy from the light source so that it is radiated into upper airways airflow.

12. The airflow treatment system as defined in claim 11, wherein any part of the system is surgically implanted within body of the user, using biocompatible material for the apparatus housing if it is a part of the system being surgically implanted.

13. The airflow treatment system as defined in claim 11, wherein the complete system is surgically implanted within body of the user, using biocompatible material for the apparatus housing.

14. The airflow treatment system as defined in claim 1, wherein the electronic control module comprises an antenna for wireless connection to an external device for transmitting and receiving information, optionally this external device may be a smartphone.

15. The airflow treatment system as defined in claim 14, wherein the electronic control module comprises an integrated computer managing the system functionality based on information received from external device, optionally this external device may be a smartphone.

16. (canceled)

17. The airflow treatment system as defined in claim 1, wherein the apparatus includes a fiber optic mat that directs the electromagnetic energy into upper airways airflow, optionally the fiber optic mat comprises a fiber optic weave.

18. The airflow treatment system as defined in claim 1, wherein the apparatus includes an optical diffuser that directs the electromagnetic energy into upper airways airflow.

19. The airflow treatment system as defined in claim 1, wherein the apparatus is powered by a wireless power transfer.
